(19) United States
(12) Patent Application Publication
(10) Pub. No.: US 2009/0326339 A1
(43) Pub. Date: Dec. 31, 2009

(54) CONNECTED HEALTHCARE DEVICES WITH REAL-TIME AND PROACTIVE CAPTURE AND RELAY OF CONTEXTUAL INFORMATION

(75) Inventor: Eric J. Horvitz, Kirkland, WA (US)

Correspondence Address: LEE & HAYES, PLLC, 601 W. RIVERSIDE AVENUE, SUITE 1400, SPOKANE, WA, 99201, US

(73) Assignee: Microsoft Corporation, Redmond, WA (US)

(21) Appl. No.: 12/147,353

(22) Filed: Jun. 26, 2008

(51) Int. Cl. A61B 5/00; G06Q 50/00

(52) U.S. Cl. 600/301; 705/3

(57) ABSTRACT

A portable healthcare device is connected to a networked healthcare service to form a connected healthcare system that is configurable to address a particular predicted or emergent healthcare condition or provisioned to address a number of emergent healthcare conditions. A portable sensor detects the predicted or emergent healthcare condition. A two-way communication channel provides instructions to the affected person or those in proximity, takes remote control of the portable healthcare device to deliver a therapeutic intervention, or facilitates a rendezvous with dispatched first responders. A buffered quantity of data that is recorded proactively can be transmitted in order to make a more accurate remote diagnosis. The remote, networked healthcare service can maintain additional information about the device or a person assigned to the device to augment the transmission, including healthcare records, contact information, configuration type of the device including therapeutic capabilities, service billing, facility location of an assigned device, etc.

### BACKGROUND

Advances continue to address the needs of persons who can experience a life-threatening condition without the benefit of an attending healthcare provider. Significant development has been made on portable clinics, medical data that can be distributed across a network, and even remotely controlled surgical instruments for performing treatments in remote, austere environments. Medical expertise in one location is leveraged to support a greater range to where the need exists. However, such solutions are affordable only to large institutional interests or in limited settings having the necessary infrastructure.

Other advances address to varying degree the need for rapid and distributed healthcare services to a larger group of untrained users. Such partial solutions require a modest cost of ownership. For example, a person who lives alone can activate a portable two-way communication device when unable to reach a traditional telephone. As another example, implantable devices have increasing capabilities for providing a therapeutic agent (e.g., defibrillation, drug dispensing, etc.). In addition, first responders have automated beacons that activate after a period of time in which a wearer fails to move. Emergent conditions that can be suffered are given a degree of mitigation.

As a further example, Automated External Defibrillators (AEDs) are becoming ubiquitous in many public and private facilities. Generally, though, a vast range of medical conditions can occur that differ from patient to patient that are difficult or expensive to address in a mass distributed portable device. Moreover, the general public has a modest amount of medical knowledge. Thus, although conventional healthcare-related devices are significantly helpful in certain situations, such devices are constrained in the automated responses provided. Achieving sufficient economies in cost and size requires limiting the parameters sensed, the computational diagnostic capabilities incorporated, and range of therapeutic actions enabled.

Thus, as our society becomes notably more mobile and connected, tragic situations continue to arise where a person experiences an emergent healthcare condition for which a therapeutic intervention is not provided within time, especially when time-critical needs arise and/or when the person is impaired or unconscious such that use of a mobile communication device (e.g., cell phone) is not utilized by the person to contact assistance.

### SUMMARY

The following presents a simplified summary of the innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

The subject innovation relates to systems and/or methods that provide advantages of portable, automated healthcare devices that can mitigate an emergent condition for a person who is remote to a traditional healthcare provider. Expertise can be remotely relayed to the healthcare device after a portable sensor detects an emergent condition. The monitoring and two-way communication can be affordably provided enabling widespread acceptance and use. Such two-way communication can include the transmittal of key physiological signs and symptoms as well as surrounding contextual cues, including recent monitored histories of contextual cues and physiology that may be continually monitored even in the absence of a concerning event. Moreover, with the expertise remotely accessed only upon a detected a need, a greater range of therapeutic capabilities can be enabled within the device, deployed upon authorization by the appropriate medical provider.

In accordance with one aspect of the subject innovation, a method is provided for expediting healthcare services to a person outside of a healthcare facility who is experiencing an emergent condition. An emergent condition of a person is sensed with sensor. The emergent condition is communicated a remote network. A two-way communication interface is provided for the person with a remote dispatcher of the wireless network.

In another aspect, an apparatus expedites healthcare services to a person outside of a healthcare facility who is experiencing an emergent condition. A portable sensor senses an emergent condition of a person. A communication module communicates the emergent condition to a wireless network. A user interface presents a two-way communication interface for the person with a remote dispatcher of the wireless network. A housing contains the portable sensor, communication module and user interface for making the apparatus readily portable.

In an additional aspect, a portable apparatus expedites healthcare services to a person outside of a healthcare facility who is experiencing an emergent condition. A location sensing component provides location data for the apparatus. A dual mode communication module accesses at least two of a group consisting of a cellular telephone communication channel, a wireless access point, and a personal access network. A user interface provides two way audio and video communication and at least local display of graphical data. A first electromagnetic sensor senses an emergent condition of a person related to cardiopulmonary function. A blood monitor senses an abnormal condition of the blood. An emergent condition component monitors and buffers the sensed location, cardiopulmonary, and blood data and for responding to a detected emergent condition by utilizing the dual mode communication module to communicate the emergent condition to a wireless network. An intervention module responds to the emergent condition component and comprises a defibrillation protocol component, a therapeutic compound dispenser, and a locator beacon. A housing contains the portable apparatus.

The following description and the annexed drawings set forth in detail certain illustrative aspects of the claimed subject matter. These aspects are indicative, however, of but a few of the various ways in which the principles of the innovation may be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 illustrates a block diagram of an exemplary system that facilitates a connected healthcare delivery to a person experiencing an emergent condition remote to healthcare providers.

FIG. 2 illustrates a block diagram of another exemplary system configurable to address a plurality of emergent conditions.

FIG. 3 illustrates a flow diagram for a methodology for emergent healthcare condition mitigation.

FIG. 4 illustrates a block diagram of an additional exemplary system that addresses a plurality of healthcare conditions, coordinating a response via a portable device.

FIG. 5 illustrates a timing diagram for a methodology for responding to a plurality of emergent healthcare conditions with a portable connected healthcare device.

FIG. 6 illustrates an exemplary networking environment, wherein the novel aspects of the claimed subject matter can be employed.

FIG. 7 illustrates an exemplary operating environment that can be employed in accordance with the claimed subject matter.

### DETAILED DESCRIPTION

A portable healthcare device is connected to a networked healthcare service, configurable to address a particular emergent healthcare condition or provisioned to address a number of emergent healthcare conditions. A portable sensor detects the emergent healthcare condition without the need for a healthcare provider, prompting connecting to remote expertise to either utilize a two-way communication channel to provide instructions to the affected person or those in proximity, to take remote control of the portable healthcare device to deliver a therapeutic intervention of nature that requires authorization from a healthcare provider, or to facilitate a rendezvous with dispatched first responders, which can be enhanced by a location sensor incorporated into the device. A buffered quantity of data can be transmitted in order to make a more accurate remote diagnosis. The remote, networked healthcare service can maintain additional information about the device or a person assigned to the device to augment the transmission, including healthcare records, contact information, configuration type of the device including therapeutic capabilities, service billing, facility location of an assigned device, etc.

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter may be practiced without these specific details. In other instances, well-known structures and devices are shown in block diagram form in order to facilitate describing the subject innovation.

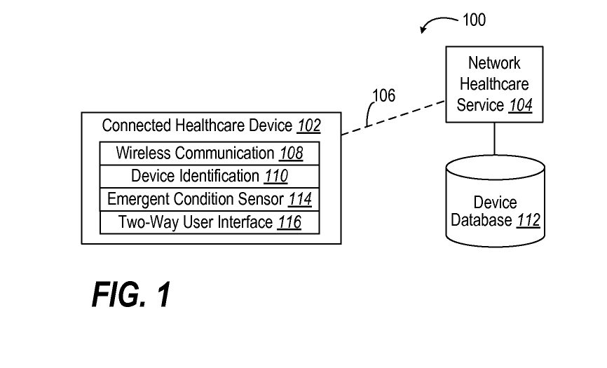

Now turning to the figures, FIG. 1 illustrates a system 100 for mitigating emergent healthcare conditions for a person remote from healthcare providers with an economical and portable healthcare device 102 that can be connected remotely to a network healthcare service 102 over a wireless air interface 104 in an exemplary aspect, although it should be appreciated that a wired interface can be used in some implementations. To that end, a wireless communication module 108 can make periodic contact with the network 104, such as to provide device operability status. Such reports with device identification provided by a stored device ID 110 can be used to infer failure when reports fail or to extrapolate location to areas without wireless coverage. The latter can trigger a follow up if reports do not resume within a preset period of time, such as referring to contact information, service setting, or chronic susceptibility healthcare information contained in a database 112 utilized by the network healthcare service 104. A portable sensor 114 for detecting an emergent healthcare condition triggers the device 102 to make a report the network healthcare service 104, opening a two-way user interface for facilitating healthcare expertise to assist the endangered person with the emergent condition.

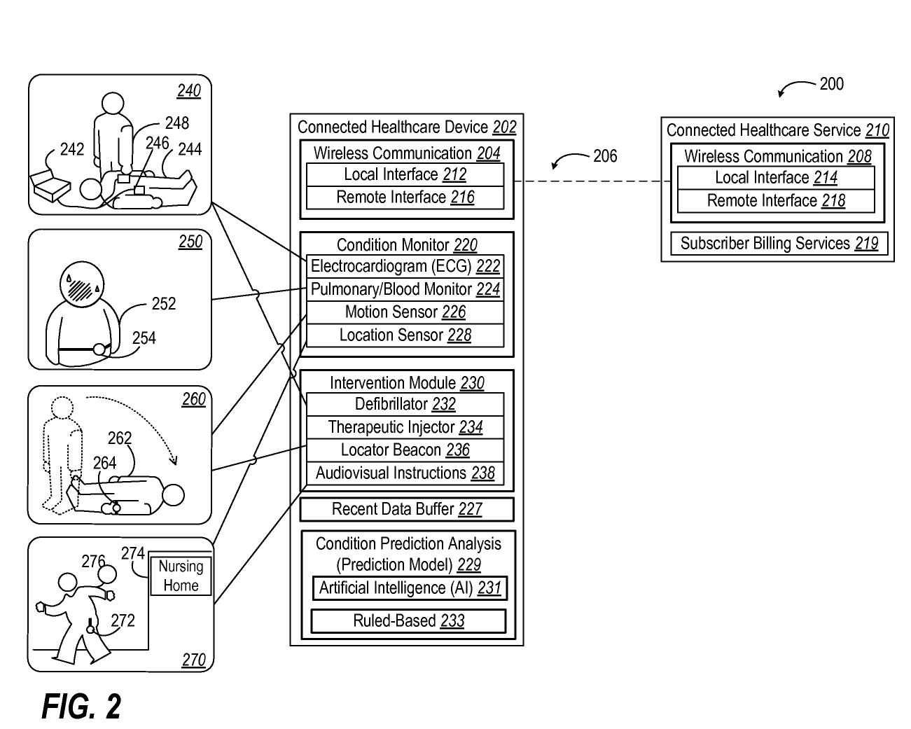

In another aspect, in FIG. 2, another exemplary system 200 includes a connected healthcare device 202 that is configurable or provisioned to address a number of emergent conditions. To that end, the device 202 that includes a wireless communication module or component 204 that connects remotely over a wireless air interface 206 to a network communication module or component 208 connected healthcare service 210, each having a respective local interface 212, 214 and remote interface 216, 218 for allowing local control and remote control to utilize the communication channel over the air interface 206.

A condition monitor 220 either persistently or upon manual activation detects the presence of an emergent healthcare condition to prompt an urgent use of the wireless communication component 204 that could also be used for routine communications regarding status. Sensing and diagnostic computation for illustrative emergent conditions are illustrated by an electrocardiogram (ECG) component 222, a pulmonary/blood monitoring component 224, a motion sensor 226, and a location sensor 228. This emergent condition monitor 220 can monitor and buffer in a recent data buffer 227 the sensed location, cardiopulmonary, and blood data and for responding to a detected emergent condition by utilizing the dual mode communication module 204 to communicate the emergent condition to the wireless network module 208. Similar sensing can be used in conjunction with a predictive model 229 for condition prediction analysis so as to predict that an emergent condition will occur within some time horizon over some threshold probability for intervening. Predictive models can be local or actually can be based on a central server so that data can be sent back over time intermittently and the learning and reasoning can occur centrally for the predictive and diagnostic models.

The data buffer 227 can for example hold certain data summaries or demographic type data for longer periods of time (e.g., regularity of exercise, statistical summaries of physiological parameters, etc.). High fidelity data capture can be for more recent data, such as the last ten minutes, in a manner similar to aircraft “black boxes”. The erasure and reuse of the storage capacity can also take advantage of remote offline storage or detecting certain data items of interest for retention in order to free up space without loss of data useful for predictions, diagnoses, etc.

The device 202 can contain the augmented processing that enhances various features, such as being part of the prediction model 229. Examples of augmenting processing include an artificial intelligence (AI) component 231 that facilitates automating one or more features in accordance with the subject invention. The subject invention (e.g., with respect to determining a present or target location, communicating location-based data and/or services . . . ) can employ various AI-based schemes for carrying out various aspects thereof. For example, a predicting an emergent condition can be trained and facilitated via an automatic classifier system and process.

A classifier is a function that maps an input attribute vector, x=(x1, x2, x3, x4, xn), to a class label class(x). A classifier can also output a confidence that the input belongs to a class, that is, f(x)=confidence(class(x)). Such classification can employ a probabilistic and/or statistical-based analysis (e.g., factoring into the analysis utilities and costs) to prognose or infer an action that a user desires to be automatically performed.

A support vector machine (SVM) is an example of a classifier that can be employed. The SVM operates by finding a hypersurface in the space of possible inputs that splits in an optimal way the triggering input events from the non-triggering events. Other classification approaches, including Naïve Bayes, Bayesian networks, decision trees, neural networks, fuzzy logic models, maximum entropy models, etc., can be employed. Classification as used herein also is inclusive of statistical regression that is utilized to develop models of priority.

As will be readily appreciated from the subject specification, the subject invention can employ classifiers that are pre-trained (e.g., via a generic training data from multiple users) as well as methods of reinforcement learning (e.g., via observing user behavior, observing trends, receiving extrinsic information). Thus, the subject invention can be used to automatically learn and perform a number of functions, including but not limited to determining, according to a predetermined criteria, whether certain motions are indicative of injury, certain geographic locations are indicative of being lost, certain cardiopulmonary or other physiological conditions are dangerously abnormal, and whether certain trends in sensed data can be predicted to exceed a normal range within a certain period of time.

Another example of prediction model 229 includes a rules-based logic component 233. In accordance with this alternate aspect, an implementation scheme (e.g., rule) can be applied to define thresholds, initiate trigger certain communication options, facilitate locating the person, etc. By way of example, it will be appreciated that the rule-based implementation can automatically define criteria thresholds whereby an analyzer component or processor can employ the thresholds to determine balance false alarms against reliable response to emergent conditions, such as providing graduations in response due to certainty in the prognoses or amount of time available for an effective response. It is to be appreciated that any of the specifications and/or functionality utilized in accordance with the subject invention can be programmed into a rule-based implementation scheme. It is also to be appreciated that this rules-based logic can be employed in addition to, or in place of, AI reasoning components.

An intervention module 230 can employ an automated response to provide a therapeutic treatment. Alternatively or in addition, the intervention module 230 can provide a capability remotely authorized for use by the connected healthcare service 210. Illustrative intervention modules 230 are depicted as a defibrillator 232, a therapeutic injector 234, locator beacon 236 to assist in reaching the device 202, and an audiovisual instruction component 238 to guide the person afflicted or bystanders to use the intervention module 230. Thus, in some aspects, the intervention module 230 responds to the predicted or currently emergent condition in response to the emergent condition monitor 220 or prediction model 229 and comprises either a warning and readying for or for initiating the defibrillation protocol component (defibrillator) 232, the therapeutic compound dispenser (injector) 234, and the locator beacon 236.

It should be appreciated with the benefit of the present disclosure that the intervention can be modulated based upon the time criticality of the prediction or detection. For example, a trend can indicate a problem such as a myocardial infarction that is imminent that could be alleviated by the patient taking an aspirin and having an ambulance contacted could be one option whereas sensed indications that the person is unconscious with falling vital signs after the onset of the myocardial infarction could illicit a more aggressive intervention protocol. Instructions could be relayed to the person or others assisting the person to ready the device in a predicted emergent condition in case it is needed for therapeutic application.

As another aspect, detection/predictions could adjust thresholds by recognizing certain circumstances. For example, a pattern of motion that correlates with elevated physiological parameters can be sensed as exercise whereas rising physiological parameters without an apparent cause can prompt a different determination.

The range of emergent conditions for which the device 202 can be configured or provisioned to address is illustrated by four vignettes. First, as depicted at 240, the healthcare device 202 comprises portable packaging 242 as a kit prepositioned or carried for use when a person 244 in the vicinity appears to be in need. In an illustrative application, an automated external defibrillator kit provides instructions and electrodes 246 for another person 248 to attach to the person in need.

Second, as depicted at 250, a person 252 wears an external healthcare device 254, which in some instances includes an implanted sensor (not shown). Chronic conditions are monitored, such as blood sugar monitoring, dissolved blood oxygen monitoring or respiration rate for chronic respiratory diseases, or multi-symptom detection for anaphylaxis shock due to environmental or food hyper allergic reaction. Intervention can include increased infusion or injection of a drug.

Third, as depicted at 260, a person 262 wears an external healthcare device 264 that monitors motion. For example, a sudden impact indicative of a fall can trigger a response. The amount of the fall can be preset to accommodate a lower threshold for an elderly user as compared to a young adult who engages in high adventure activities. The motion detector can be triggered by an inappropriately long period of time in one location that can indicate an injury preventing ambulatory movement. An enabling sensor (not shown) can confirm continued attachment to the person to indicate a lesser condition of failure to wear the device 264.

Fourth, as depicted at 270, a person wears a healthcare device 272 that detects location, such as a geographic location with reference to a global positioning system (GSP) or relative to a facility 274, such as defined by an electronic fence or radio frequency identifier (RFID) system, etc. Alternatively or in addition, the connected healthcare service 210 can use signal direction finding and received power measurement to estimate a location for the device 272. Thus, when an incompetent person 276 (e.g., child, mentally deficient adult, etc.) leaves a permissible area, the device 272 triggers intervention such as verbal instructions, map to guide the person back, can automatically or remotely be triggered beacon to emanate a humanly perceptible or sensor perceptible beacon to guide bystanders or certain first responders to the assistance of the person 276

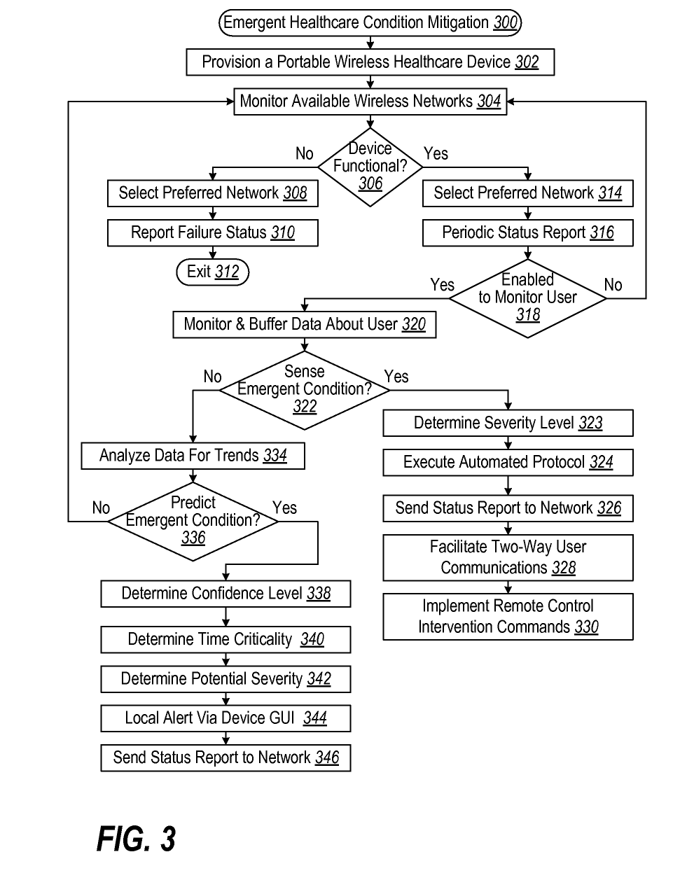

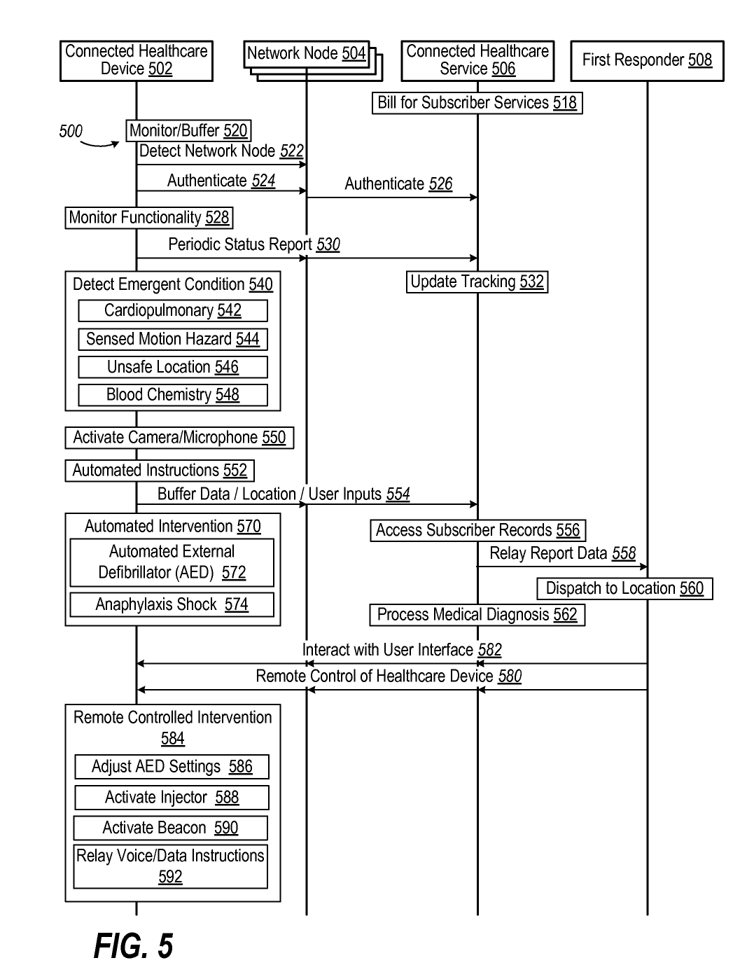

FIGS. 3 and 5 illustrate methodologies and/or flow diagrams in accordance with the claimed subject matter. For simplicity of explanation, the methodologies are depicted and described as a series of acts. It is to be understood and appreciated that the subject innovation is not limited by the acts illustrated and/or by the order of acts. For example, acts can occur in various orders and/or concurrently, and with other acts not presented and described herein. Furthermore, not all illustrated acts may be required to implement the methodologies in accordance with the claimed subject matter. In addition, those skilled in the art will understand and appreciate that the methodologies could alternatively be represented as a series of interrelated states via a state diagram or events. Additionally, it should be further appreciated that the methodologies disclosed hereinafter and throughout this specification are capable of being stored on an article of manufacture to facilitate transporting and transferring such methodologies to computers. The term article of manufacture, as used herein, is intended to encompass a computer program accessible from any computer-readable device, carrier, or media.

With reference to FIG. 3, a methodology 300 for emergent healthcare condition mitigation begins in block 302 with configuring or provisioning a portable wireless healthcare device. Once enabled, the healthcare device monitors for available wireless networks in block 304, for example taking advantage of lower cost or higher bandwidth wireless channels when available or performing reporting sufficient for assignment to a cellular radio access node, etc. For devices capable of self test, in block 306 a determination is made as to whether the device is function (e.g., sensors are operable, power supply is adequate, computational components test as functional, intervention capabilities are stocked, etc.). If not, the preferred available network is select in block 308 and the failure status is reported in block 310 and methodology 300 exits in block 312. For example, an AED kit prepositioned in a facility can report itself for routine maintenance or replacement. As another example, a device worn by a user can be reported as failed or of an imminent failure prompting a communication to the person or a caregiver for expedited replacement. Otherwise, the device can periodically report a functional status by selecting a preferred available network in block 314 and making a periodic status report in block 316.

A determination is made as to whether monitoring for one or more emergent conditions has been enabled in block 318, and if not processing returns to block 304. If so, in block 320 the device continues to monitor for the emergent condition and to advantageously buffer a recent period of data. A determination is made in block 322 as to whether the emergent condition is sensed. If sensed, then a severity level for the emergent condition is determined, such as via a cross reference (block 323), and an automated intervention protocol can be executed in accordance with the severity level in block 324. A status report can be sent to the network in block 326. Two-way user communication is facilitated in block 328. Remote control intervention commands are implemented in block 330.

If a current emergent condition was not sensed in block 322, then the buffered data and patient characterization data if available (e.g., setting for susceptibility like asthma, heart disease, etc.) are analyzed for trends (block 334). A determination is made as to whether an emergent condition can be predicted in block 336. A confidence level in the prediction is determined in block 338. The time criticality until the emergent condition is predicted to occur is determined in block 340. The potential severity of the emergent condition is determined in block 342. Based on these determinations, a local alert can be made via the device GUI in block 344, such to alert the person or bystanders to ready the device for therapeutic action, to take actions such as steps appropriate for heat exhaustion or heat stroke, etc. In block 346, a status report is made to the network, which can include alerting a first responder.

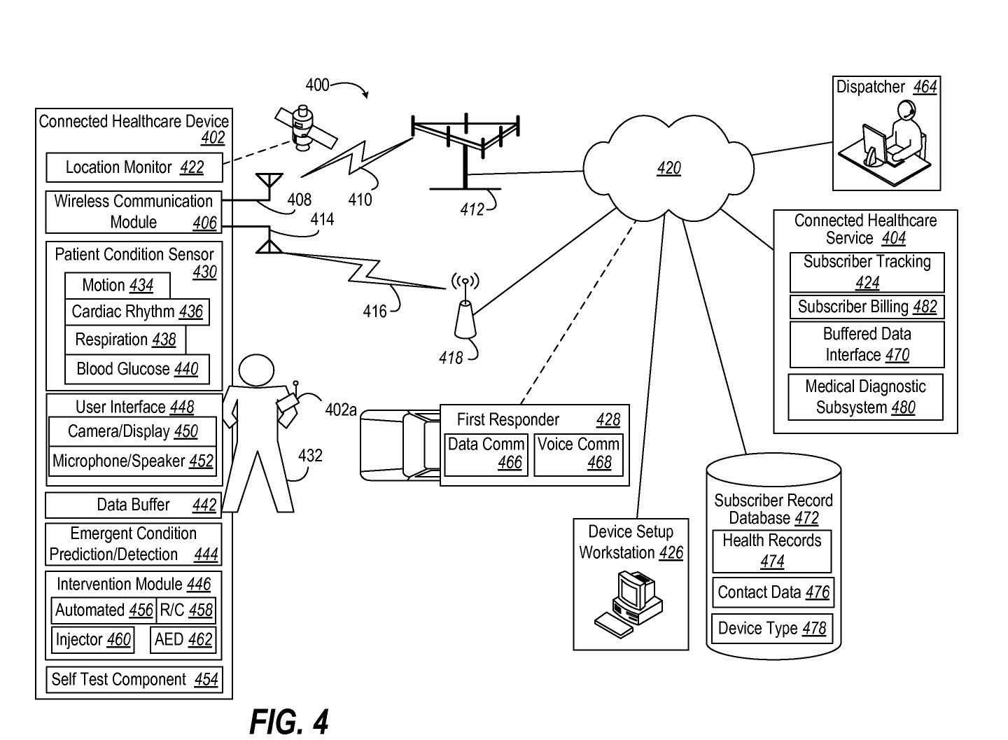

In FIG. 4, a connected healthcare system 400 includes a connected healthcare device 402 configured for specific or provisioned for a number of emergent conditions either autonomously or alternatively in addition delivered by a connected healthcare service 404 remote to the device 402. A wireless communication module 406 of the healthcare device 402 can advantageously be a dual channel depicted as a first transceiver 408 communicating via a wireless communication channel 410 to a cellular telephone radio access node (RAN) 412 and a second transceiver 414 communicating via a wireless communication channel 416 to an access point 418 in order to reach the healthcare service 404 via a private or public network 420 (e.g., Internet, publicly switched telephone network (PSTN), etc.).

The connected healthcare device 402 can include a location monitor 422 that can determine the location of the device 402 geographically or relative to a particular network or facility reference signal. Alternatively or in addition, the connected healthcare service 404 includes a subscriber tracking component 424 that has an assigned location for the device 402, such as user input via a device setup workstation 426, to expedite dispatching of a first responder 428 as necessary to the location of the device 402. For mobile applications, this subscriber tracking component 424 can retain recent location reports, triangulation from a network node in contact with the device, or extrapolate from last known locations.

One or more patient condition sensors 430 monitor a user 432 depicted as having a wearable connected healthcare device 402a. Illustrative sensors 430 are depicted as a motion sensor 434, a cardiac rhythm sensor 436, a respiration monitor 438, and a blood glucose sensor 440. The data readings are buffered in a data buffer 442. An emergent condition prediction/detection component 444 can detect a pattern or threshold in the sensed data that is indicative of an emergent condition warranting alerting of the user 432, alerting of the health care service 404, and/or activating an intervention module 446. The data buffered can include audio and video recordings from a user interface 448 depicted having a camera/display component 450 and a microphone/speaker component 452. The user interface 448 can provide a means for automated instructions to the user 432 or a bystander assisting the user by employing the connected healthcare device 402. Buffering of audiovisual information can assist the first responder 428 in locating the person 432. A self-test component 454 can alert the user 432 or the service 404 of a failure or impending failure or prevent a false reporting of an emergent condition. The intervention module 446 can be equipped to respond to automated commands 456 or to remote control (R/C) commands 458 to take an action, such as activating a drug infuser or injector 460 or to use an automatic external defibrillator (AED) 462.

The healthcare service 404 augments the capabilities of the healthcare device 402 by allowing a dispatcher 464 or the first responders 428 to interact via a data communication module 466 or a voice communication module 468. The healthcare service 404 can also utilize buffered data interface 470, additional data from a subscriber record database 472, such as health records 474, contact data 476, and device type 478 to further inform the dispatcher 464 or first responders 428. A medical diagnostic subsystem 480 can apply a larger institutional processing capability to the data than available at the device to advise the first responders 428 or to remotely control the intervention module 446. The healthcare service 404 can also respond to normal subscription periods or to services delivered in response to a reported emergent condition to utilize a subscribing billing component 482.

With reference to FIG. 5, a methodology 500 is depicted for interactions between a connected healthcare device 502, a network node 504, a connected healthcare service 506, and a first responder 508 to detect and mitigate emergent conditions. The Connected Healthcare Service 506 can manage a subscription-based service that is purchased in conjunction with a plurality of healthcare devices 502 (block 518). The service could be provided as part of the purchase or lease price of the device 502. As depicted at 520, the connected healthcare device 502 monitors a sensor and buffers the received data for a person. In order to increase mobility, as depicted at 522, the device 502 detects a network node 522 when available. Authentication is made between the device 502 and the service 506 via the network node 504 as depicted at 524, 526. The device 502 monitors its functionality as depicted at 528. As depicted at 530, a periodic status report is made from the device 502 to the service 506 so the service can update tracking as depicted at 532, extrapolate location, determine the need to inquire into outages, respond to failures, etc.

A determination is made as to one or more emergent conditions has been detected at 540, such as a cardiopulmonary abnormality at 542, a sensed motion hazard at 544, an unsafe location at 546, and abnormal blood chemistry at 548.

The device 502 takes mitigating actions illustrated by activating a camera/microphone at 550 to alert the user or bystanders as depicted at 552, to capture situation data to forward to the service 506 and first responders 508. The buffered sensed data and audiovisual and advantageously location information for the device are transmitted to the service 506 as depicted at 554.

Remotely controlled intervention can be facilitated by the service 506. For instance, subscriber records can be accessed at block 556. This supplemental data as well as the data received from the device 502 can be relayed to the first responder 508 to assist their prioritizing and expeditious delivery of appropriate assets, depicted as being dispatched to location in block 560. The service 506 can process the accumulated data using institutional resources, either automated or human or both, to generate a more through medical diagnosis, as depicted at 562.

Automated intervention at the device 502 can occur as depicted in block 570, illustrated by an automated external defibrillator at 572 and an anaphylaxis shock treatment at 574. The service can interact with the user or assisting bystander via a two-way communication channel as depicted at 580 to give status for arrival of the first responders 508 or other information.

The intervention by the service 506 can entail taking remote control of the healthcare device as depicted at 582 to utilize therapeutic capabilities of the device 502. These actions are illustrated by adjusting AED settings at 586, such as increasing a charge or number of charged given. A therapeutic infuser or injector can be activated to give an amount of drug as depicted at 588. A humanly perceptive or machine detected locator beacon can be activated as depicted at 590 to alert bystanders or first responders as to the location of device 502. The remote control can entail relaying voice/data instructions as depicted at 592.

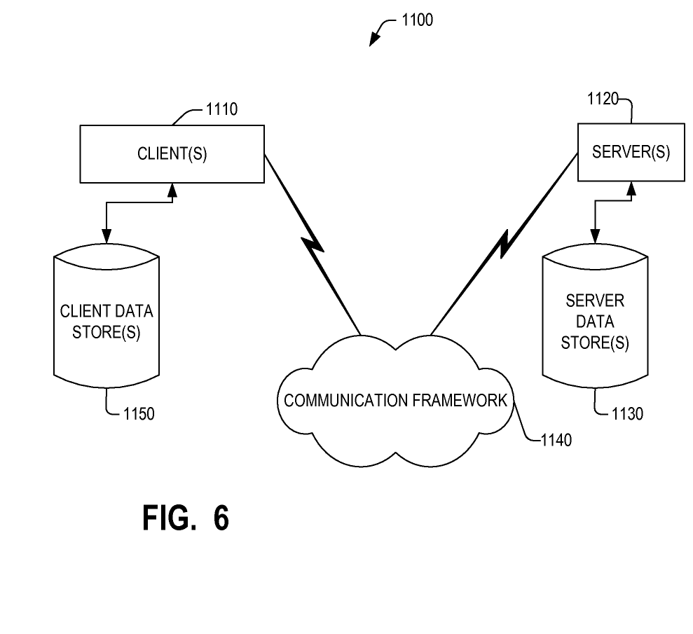

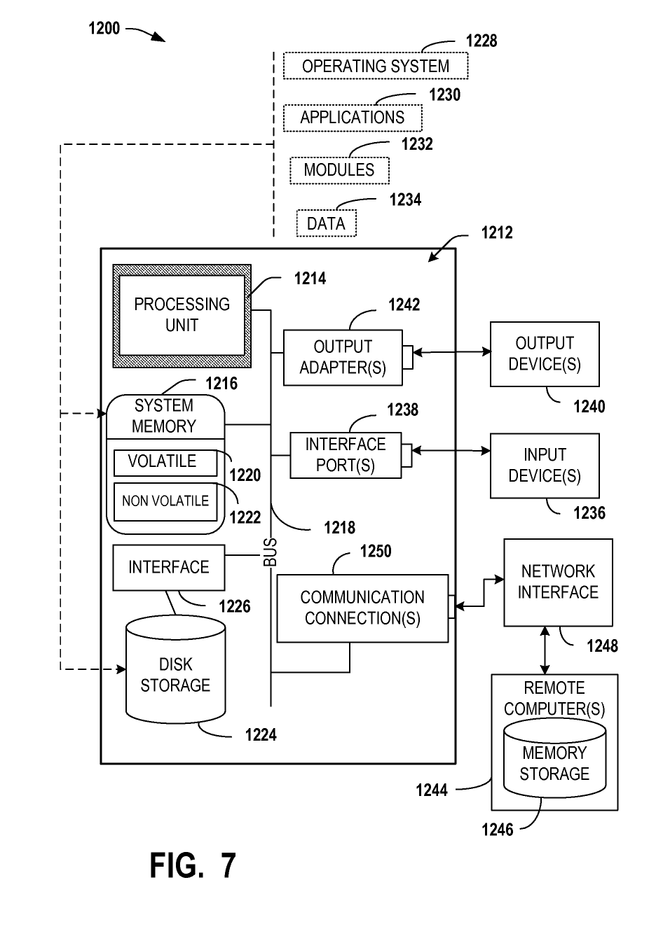

In order to provide additional context for implementing various aspects of the claimed subject matter, FIGS. 6-7 and the following discussion is intended to provide a brief, general description of a suitable computing environment in which the various aspects of the subject innovation may be implemented. For example, a counselor component that facilitates automatically generating questions to ask a doctor during an appointment, as described in the previous figures, can be implemented in such suitable computing environment. While the claimed subject matter has been described above in the general context of computer-executable instructions of a computer program that runs on a local computer and/or remote computer, those skilled in the art will recognize that the subject innovation also may be implemented in combination with other program modules. Generally, program modules include routines, programs, components, data structures, etc., that perform particular tasks and/or implement particular abstract data types.

Moreover, those skilled in the art will appreciate that the inventive methods may be practiced with other computer system configurations, including single-processor or multi-processor computer systems, minicomputers, mainframe computers, as well as personal computers, hand-held computing devices, microprocessor-based and/or programmable consumer electronics, and the like, each of which may operatively communicate with one or more associated devices. The illustrated aspects of the claimed subject matter may also be practiced in distributed computing environments where certain tasks are performed by remote processing devices that are linked through a communications network. However, some, if not all, aspects of the subject innovation may be practiced on stand-alone computers. In a distributed computing environment, program modules may be located in local and/or remote memory storage devices.

FIG. 6 is a schematic block diagram of a sample-computing environment 1100 with which the claimed subject matter can interact. The system 1100 includes one or more client(s) 1110. The client(s) 1110 can be hardware and/or software (e.g., threads, processes, computing devices). The system 1100 also includes one or more server(s) 1120. The server(s) 1120 can be hardware and/or software (e.g., threads, processes, computing devices). The servers 1120 can house threads to perform transformations by employing the subject innovation, for example.

One possible communication between a client 1110 and a server 1120 can be in the form of a data packet adapted to be transmitted between two or more computer processes. The system 1100 includes a communication framework 1140 that can be employed to facilitate communications between the client(s) 1110 and the server(s) 1120. The client(s) 1110 are operably connected to one or more client data store(s) 1150 that can be employed to store information local to the client(s) 1110. Similarly, the server(s) 1120 are operably connected to one or more server data store(s) 1130 that can be employed to store information local to the servers 1120.

With reference to FIG. 7, an exemplary environment 1200 for implementing various aspects of the claimed subject matter includes a computer 1212. The computer 1212 includes a processing unit 1214, a system memory 1216, and a system bus 1218. The system bus 1218 couples system components including, but not limited to, the system memory 1216 to the processing unit 1214. The processing unit 1214 can be any of various available processors. Dual microprocessors and other multiprocessor architectures also can be employed as the processing unit 1214.

The system bus 1218 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1216 includes volatile memory 1220 and nonvolatile memory 1222. The basic input/output system (BIOS), containing the basic routines to transfer information between elements within the computer 1212, such as during start-up, is stored in nonvolatile memory 1222. By way of illustration, and not limitation, nonvolatile memory 1222 can include read only memory (ROM), programmable ROM (PROM), electrically programmable ROM (EPROM), electrically erasable programmable ROM (EEPROM), or flash memory. Volatile memory 1220 includes random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM).

Computer 1212 also includes removable/non-removable, volatile/non-volatile computer storage media. FIG. 7 illustrates, for example, disk storage 1224. Disk storage 1224 includes, but is not limited to, devices like a magnetic disk drive, floppy disk drive, tape drive, Jaz drive, Zip drive, LS-100 drive, flash memory card, or memory stick. In addition, disk storage 1224 can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 1224 to the system bus 1218, a removable or non-removable interface is typically used such as interface 1226.

It is to be appreciated that FIG. 7 describes software that acts as an intermediary between users and the basic computer resources described in the suitable operating environment 1200. Such software includes an operating system 1228. Operating system 1228, which can be stored on disk storage 1224, acts to control and allocate resources of the computer system 1212. System applications 1230 take advantage of the management of resources by operating system 1228 through program modules 1232 and program data 1234 stored either in system memory 1216 or on disk storage 1224. It is to be appreciated that the claimed subject matter can be implemented with various operating systems or combinations of operating systems.

A user enters commands or information into the computer 1212 through input device(s) 1236. Input devices 1236 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1214 through the system bus 1218 via interface port(s) 1238. Interface port(s) 1238 include, for example, a serial port, a parallel port, a game port, and a universal serial bus (USB). Output device(s) 1240 use some of the same type of ports as input device(s) 1236. Thus, for example, a USB port may be used to provide input to computer 1212 and to output information from computer 1212 to an output device 1240. Output adapter 1242 is provided to illustrate that there are some output devices 1240 like monitors, speakers, and printers, among other output devices 1240, which require special adapters. The output adapters 1242 include, by way of illustration and not limitation, video and sound cards that provide a means of connection between the output device 1240 and the system bus 1218. It should be noted that other devices and/or systems of devices provide both input and output capabilities such as remote computer(s) 1244.

Computer 1212 can operate in a networked environment using logical connections to one or more remote computers, such as remote computer(s) 1244. The remote computer(s) 1244 can be a personal computer, a server, a router, a network PC, a workstation, a microprocessor based appliance, a peer device or other common network node and the like, and typically includes many or all of the elements described relative to computer 1212. For purposes of brevity, only a memory storage device 1246 is illustrated with remote computer(s) 1244. Remote computer(s) 1244 is logically connected to computer 1212 through a network interface 1248 and then physically connected via communication connection 1250. Network interface 1248 encompasses wire and/or wireless communication networks such as local-area networks (LAN) and wide-area networks (WAN). LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, Digital Subscriber Lines (DSL), WiMax, and emerging wide area wireless networks.

Communication connection(s) 1250 refers to the hardware/software employed to connect the network interface 1248 to the bus 1218. While communication connection 1250 is shown for illustrative clarity inside computer 1212, it can also be external to computer 1212. The hardware/software necessary for connection to the network interface 1248 includes, for exemplary purposes only, internal and external technologies such as, modems including regular telephone grade modems, cable modems and DSL modems, ISDN adapters, and Ethernet cards.

What has been described above includes examples of the subject innovation. It is, of course, not possible to describe every conceivable combination of components or methodologies for purposes of describing the claimed subject matter, but one of ordinary skill in the art may recognize that many further combinations and permutations of the subject innovation are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable medium having computer-executable instructions for performing the acts and/or events of the various methods of the claimed subject matter.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein but generally known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

1. A method comprising:
affixing a wearable healthcare device to a person, the wearable healthcare device including a plurality of sensors for sensing physiological parameters of the person;
sensing, by the healthcare device, the plurality of physiological parameters of the person;
analyzing the sensed physiological parameters by employing a classifier that is pre-trained via generic training data obtained from multiple users, the classifier further considering observed cast behavior of the person for predicting whether the sensed physiological parameters are indicative of an emergent condition of the person;
when the sensed physiological parameters support a prediction that the emergent condition will occur within a predetermined time horizon over a predetermined threshold probability for intervening, communicating the emergent condition to a remote network; and
presenting a two-way communication interface for the person with a remote dispatcher of the remote network.

2. The method of claim 1, further comprising communicating a device identification to the remote network for correlating with additional data associated with the device, wherein the additional data is selected from a group consisting of location data, assigned user healthcare records, device configuration data, and a therapeutic capability of the device.

3. The method of claim 2, further comprising recognizing a pattern of motion of the person that is indicative of exercise and adjusting the predetermined threshold probability for intervening.

4. The method of claim 1, further comprising sensing an emergent condition of an abnormal electrocardiogram.

5. The method of claim 4, further comprising performing an automated external defibrillation.

6. The method of claim 5, further comprising responding to remote control to adjust settings of the automated external defibrillation.

7. The method of claim 1, further comprising sensing the emergent condition of pulmonary distress.

8. The method of claim 1, further comprising sensing the emergent condition of unconsciousness.

9. The method of claim 1, further comprising sensing the emergent condition of abnormal blood sugar level.

10. The method of claim 1, further comprising sensing motion of a person wearing the sensor and detecting the emergent condition of falling indicative of an injury.

11. The method of claim 10, further comprising remotely triggering a location beacon via the two-way interface.

12. The method of claim 1, further comprising sensing the emergent condition of the person at a location outside of a defined area as defined by a radio frequency identifier system.

13. The method of claim 1, further comprising buffering data of the sensed physiological parameters prior to detecting the emergent condition, wherein the buffering comprises maintaining recent data in at a first level of detail for a first period of time, and maintaining summary data at a lower level of detail for longer period of time than the first period of time.

14. The method of claim 13, further comprising sensing the emergent condition by analyzing the buffered data to detect a trend and predicting the trend to become the emergent condition.

15. The method of claim 14, further comprising predicting the trend based upon artificial intelligence augmented processing.

16. The method of claim 14, further comprising predicting the trend based upon rule based augmented processing.

17. The method of claim 14, further comprising selecting a communication and therapeutic response as a function of confidence and time criticality of the prediction.

18. The method of claim 14, further comprising notifying the person via a local user interface.

19. The method of claim 13, further comprising uploading the buffered data to the remote network.

20. The method of claim 1, further comprising communicating the emergent condition with a wireless network.

21. The method of claim 20, further comprising:
monitoring a plurality of disparate wireless radio access technologies; and
periodically reporting status via a preferred accessible wireless radio access technology.

22. The method of claim 21 further comprising monitoring at least two wireless radio access technologies selected from a group consisting of a data packet wireless access point, a cellular radio access node, and a personal access network.

23. The method of claim 1, further comprising receiving a remote control command to dispense a therapeutic compound to the person.

24. The method of claim 1, further comprising presenting the two-way communication interface for the person to communicate via voice communication with a remote dispatched first responder en route to the person.

25. The method of claim 1, further comprising presenting both audio and video as the two-way communication interface.

26. The method of claim 1, further comprising sensing the emergent condition of the person with a portable sensor.

27. The method of claim 1 further comprising sensing the emergent condition with a plurality of sensors selected from a group consisting of a motion sensor, a location sensor, a cardiac sensor, and a pulmonary blood sensor.

28. The method of claim 1, further comprising billing a subscriber associated with a healthcare device comprising the sensor and the two-way communication interface.

29. A method comprising:
affixing a wearable healthcare device to a person, the wearable healthcare device including a plurality of sensors for sensing physiological parameters of the person;
sensing, by the healthcare device, the plurality of physiological parameters of the person;
analyzing the sensed physiological parameters by employing a classifier that is pre-trained via generic training data obtained from multiple users, the classifier further considering observed past behavior of the person for predicting whether the sensed physiological parameters are indicative of an emergent condition of the person;
when the sensed physiological parameters support a prediction that the emergent condition will occur within a predetermined time horizon over a predetermined threshold probability for intervening, communicating the emergent condition to a remote network;
presenting a two-way communication interface for the person to communicate via voice communication with a first responder traveling en route to the person in response to the emergent condition.

30. A portable apparatus comprising:
a location sensing component;
a dual mode communication module for accessing at least two of a group consisting of a cellular telephone communication channel, a wireless access point, and a personal access network;
a user interface for two way audio and video communication and at least local display of graphical data;
a first electromagnetic sensor for sensing an emergent condition of a person related to cardiopulmonary function;
a blood monitor for sensing an abnormal condition of the blood;
an emergent condition component for monitoring and buffering the sensed location, cardiopulmonary, and blood data and for analyzing the sensed location, cardiopulmonary, and blood data by employing a classifier that is pre-trained via generic training data obtained from multiple users, the classifier further considering observed past behavior of the person for predicting whether the sensed location, cardiopulmonary, and blood data are indicative of an emergent condition of the person, wherein when the sensed location, cardiopulmonary, and blood data support a prediction that the emergent condition will occur within a predetermined time horizon over a predetermined threshold probability for intervening, the emergent condition component responds to the detected emergent condition by communicating the emergent condition to a wireless network via the dual mode communication module;
an intervention module responsive to the emergent condition component and comprising a defibrillation protocol component, a therapeutic compound dispenser, and a locator beacon; and
a housing containing the portable apparatus.
